(19) United States
(12) Patent Application Publication
(10) Pub. No.: US 2011/0178514 A1
(43) Pub. Date: Jul. 21, 2011

(54) Cryosurgical Instrument Insulating System

(76) Inventors: Alexander Levin, Binyamina (IL); Didier Toubia, Raanana (IL); Miron Kaganovich, Haifa (IL); Kalman Gruzinsky, Carmiel (IL)

(21) Appl. No.: 13/000,020

(22) PCT Filed: Jun. 18, 2009
(86) PCT No.: PCT/IB09/52615

§ 371 Date: Mar. 31, 2011

Related U.S. Application Data
(60) Provisional application No. 61/073,415, filed on Jun. 18, 2008.

(51) Int. Cl. A61B 18/02

(52) U.S. Cl. 606/23

(57) ABSTRACT

A device, system and method for controlling the temperature differential between the material within the cryosurgical instrument and the external temperature of the shaft, apart from at the cryotip itself.

### FIELD OF THE INVENTION

The present invention is of a device, system and method for thermal insulation for a cryosurgical instrument and in particular, for such a device, system and method for maintaining a controlled external temperature for at least a portion of the probe shaft.

### BACKGROUND OF THE INVENTION

Cryoprobes (rigid cryosurgical instruments) frequently experience problems of temperature control, particularly with regard to maintaining temperature differential between the contents of the cryoprobe, which are very cold, and the outer shaft, which is desirably maintained at a higher temperature, outside of the tip itself. The outer shaft is in contact with body tissues which may be damaged by excessively cold temperatures, as only the portion of the body tissues which are in contact with the cryotip should be frozen.

At the same time the closed distal end (cryotip) of such a probe must provide in many cases high specific freezing capacity at sufficiently low temperatures.

Various attempted solutions to this problem have been provided with regard to thermal insulation of lateral non-operating walls of cryosurgical instruments.

For example, U.S. Pat. No. 3,971,383 proposes a cryogenic surgical instrument with a coaxial assembly of flexible lumens; the inner lumen is connected to a supply of cryogenic liquid, and the space between the outer wall of the inner lumen and the next lumen forms a return line for evaporated cryogenic liquid which is vented to the atmosphere. The space between the outermost one of the coaxial lumens and the intermediate lumen contains a gas, such as normal butane, serving for thermal insulation of the inner and intermediate lumens.

U.S. Pat. No. 5,573,532 describes a cryosurgical instrument, which comprises lumens of cryogenic fluid supply and return of cryogenic fluid vapors; these lumens are situated concentrically and the return lumen is sealed with a cryotip. The patent teaches vacuum insulation of the return lumen. Such a construction is very expensive and has low reliability. Besides, this vacuum insulation limits flexibility of the probe, especially when it has significant length and is used as a catheter.

U.S. Pat. No. 5,674,218 describes a cryosurgical instrument, a system and method of cryosurgery. According to this patent a cryogenic liquid (preferably, liquid nitrogen) is initially sub-cooled below its normal boiling point and then it is supplied into the open proximal end of the internal supply line. The outer lumen of the cryosurgical instrument is provided with active vacuum insulation with drawbacks as described above.

U.S. Pat. No. 7,288,089 describes an enhanced method and device intended to treat atrial fibrillation or inhibit or reduce restenosis following angioplasty or stent placement. A balloon-tipped catheter is disposed in the area treated or opened through balloon angioplasty immediately following angioplasty. The balloon, which can have a dual balloon structure, may be delivered through a guiding catheter and over a guidewire already in place. A fluid such as a perfluorocarbon flows into the balloon to freeze the tissue adjacent the balloon, this cooling being associated with reduction of restenosis. However, this construction is only useful for applications involving a balloon, such as restenosis.

U.S. Pat. No.7,273,479 describes methods and systems which are applied for cooling an object with a cryogen having a critical point defined by a critical-point pressure and a critical-point temperature. The pressure of the cryogen is raised above a pressure value determined to provide the cryogen at a reduced molar volume that prevents vapor lock. Thereafter, the cryogen is placed in thermal communication with the object to increase a temperature of the cryogen along a thermodynamic path that maintains the pressure greater than the critical-point pressure for a duration that the cryogen and object are in thermal communication. Unfortunately this system requires the application of additional high pressure.

U.S. Pat. No. 6,562,030 describes a cryocatheter, which includes a catheter body defining a coolant flow path, a catheter tip exposed to the coolant flow path, and a heating element associated with the catheter tip. The heating element can be disposed entirely or partially within the catheter tip. Alternatively, the heating element can be external to the catheter tip. The heating element can include an electrically resistive element. However, it should be noted that in any case the heating element is an active electrical element which has disadvantages.

US patent application No. 20070276360 discloses a cryosurgical catheter which is heated in order to prevent its freezing within the lumen of an endoscope. The catheter is to be used with an endoscope to perform cryoablation on an internal tissue; e.g., the esophagus. Electric conductivity to produce heat employs an electrical conductive coating on the catheter, with the previously noted disadvantages. Also, disclosed is a fitting for use with a catheter comprising both a connection for receiving gas and an electrical connection; again note that electrical power is required for the heating element.

In addition, U.S. Pat. Nos. 6,182,666, 6,095,149, 5,906,612, 5,899,897, 5,658,276 describe different versions of application of electrical heating elements for thermal insulation of untreated tissue, all of which require active electrical elements which are disadvantageous.

U.S. Pat. Nos. 5,910,104 and 6,457,212 describe the application of thermo-insulating disposable sheaths, which are situated on shafts of cryosurgical instruments and which are not suitable for most cryosurgical applications.

### SUMMARY OF THE INVENTION

The background art does not teach or suggest a simple and inexpensive mechanism for maintaining a controlled temperature differential between material inside a cryosurgical instrument and the external temperature of the shaft, away from the tip.

The present invention overcomes these drawbacks of the background art, by providing a device, system and method for controlling the temperature differential between the material within the cryosurgical instrument and the external temperature of the shaft, apart from at the cryotip itself.

A cryosurgical instrument and its accessory system, according to some embodiments, are based on application of cryogen liquids with sufficiently low boiling temperatures at pressures in the interval from below one atmosphere through several atmospheres. It should be noted that mixtures of some gases can be applied as the cryogen and/or as thermo-insulating gas.

In other embodiments, the proposed cryosurgical instrument operates according to the Joule-Thomson principle, which includes expansion of highly pressurized gas flowing via a distal orifice installed on the distal end of the central feeding tube and application of a miniature built-in counter-flow heat exchanger.

Preferably a cryosurgical instrument according to the present invention comprises an external elongated shaft; a central feeding lumen positioned in the external elongated shaft for receiving a cryogen; a cryotip, which is joined with the distal edge of the external elongated shaft; an intermediate lumen positioned coaxially between the central feeding lumen of the external elongated shaft and joined with the external elongated shaft by its distal and proximal flanges, which features a fluid medium for controlling the temperature differential between the external shaft and the intermediate lumen.

There is an additional active controller for controlling the temperature differential between the external shaft and the intermediate lumen, termed herein a temperature controller, which preferably comprises an outer coating on a significant section of the external shaft with a layer of material with very high thermal conductivity, for example, with a thick diamond film obtained by CVD (chemical vapor deposition) process. The proximal section of the diamond coating should be heated by an outer heating source installed on a handle of the cryosurgical instrument. An electrical inductor, electrical heater or heating gas can optionally serve as such outer heating source.

In another embodiment, the internal middle section of the shaft comprises a tubular piece from metal or another material with very high specific thermal conductivity, for example, copper, silver, graphite or the like, or a combination thereof. The outer side of the shaft is protected with a tubular piece from a metal, which is compatible with human tissue (stainless steel, titanium or the like, for example). The internal distal section of the shaft is preferably fabricated from a metal tubular piece (or pieces) with relatively low specific thermal conductivity (stainless steel, titanium or the like for example) in order to minimize propagation of a formed ice ball in the backward direction. The internal proximal section of the shaft is a tubular piece from a material with relatively low specific thermal conductivity, such as a metal for example; this minimizes a flux of lower temperatures in the axial direction of the cryosurgical instrument.

In some embodiments, the instrument comprises two portions which are connected through a quick coupling connection, featuring a male unit and a female unit; the former preferably is connected to the portion with the cryotip, while the latter is in contact with body tissue. For such an embodiment, the material with low specific thermal conductivity is preferably installed so as to minimize the flux of lower temperatures from the male unit to the female unit.

In a preferred embodiment, the heating means features a gas with high relative humidity; this allows to achieve very high heat transfer coefficient for heating the middle section of the external shaft and, additionally, to heat this middle section of the shaft up to a place of penetration of the cryosurgical instrument into a tissue.

In another preferred embodiment the heating gas has low relative humidity or a high level of dryness (without wishing to be limited by a single hypothesis, it prevents formation of microorganism colonies in a passageways of the heating gas).

In a further embodiment of the present invention, there is a displaceable cap, which can be slidingly shifted along the external shaft of the cryosurgical instrument. In its initial position, the cap is adjacent to a bushing with nozzles providing the heating gas and it is joined with this bushing by a set of small magnets, which are installed on the bushing and the cap itself. After penetration of the cryosurgical instrument into a tissue, the cap is shifted until it contacts with the site of tissue penetration of the cryosurgical instrument. In such a way, the cap protects the tissue from contacting the gaseous heating jets. Among other advantages, this protection obviates the need for sterilizing the heating gaseous medium supplied into the nozzles.

After termination of the cryosurgical procedure, a surgeon or other medical personnel can return the cap to its initial location and then can remove the cryosurgical instrument from the tissue.

In other embodiments of the present invention, the heating means are realized as an electrical heating element installed in the female unit of the quick coupling.

This electrical heating element is separated preferably from the other parts of the female quick coupling unit by a ring from a material with very low thermal conductivity; which may optionally comprise, for example, foamed polyurethane.

In a further embodiment, the heating means is a fluid medium (gaseous or liquid), which flows via an annular chamber in a bushing, which is provided with an inlet and outlet connections.

This bushing is separated from the female unit of the quick coupling of the cryosurgical instrument by a ring from a material with very low thermal conductivity; which may optionally comprise, for example, foamed polyurethane.

The distal end of the central feeding lumen is preferably terminated by an inlet connection for receiving cryogen, while the distal section of the elongated external shaft, which bounds the internal space between the central feeding lumen and this shaft, is preferably provided with an outlet connection for permitting the cryogen gas to be exhausted out. A proximal section of the elongated external shaft, which bounds the internal space with the intermediate lumen, is provided with an inlet connection. In another embodiment, this last inlet connection can serve alternatively as an inlet and outlet connection.

### BRIEF DESCRIPTION OF THE DRAWINGS

FIG. 1a is an axial cross-section of a cryosurgical instrument according to the present invention with heating an external shaft by streams of gaseous medium;

FIG. 1b is an axial cross-section of an external shaft with a middle internal tubular piece from a material with high specific thermal conductivity.

FIG. 1c is an axial cross-section of an external shaft with a middle outer coating by a thick diamond film.

FIG. 2 is an axial cross-section of the cryosurgical instrument with heating the external shaft by an outer electrical coil.

FIG. 3 is an axial cross-section of the cryosurgical instrument with heating the external shaft by a warming fluid medium flowing via an annular chamber.

FIG. 4a is an axial cross-section of the cryosurgical instrument, which is assembled from a disposable unit comprising an external shaft, a cryotip and a central feeding lumen; and a permanent unit, which comprises a connection element and an elongated electrical heater installed on a cylindrical elongated thermal insulator.

FIG. 4b is an axial cross-section of the disposable unit of FIG. 4a.

FIG. 4c is an axial cross-section of the permanent unit of FIG. 4a.

FIGS. 5A-5C relate to an exemplary, non-limiting embodiment with an electrical heater.

FIG. 6 shows another non-limiting, exemplary embodiment with an elongated vacuum insulated chamber applied instead of the electrical heater.

### DESCRIPTION OF PREFERRED EMBODIMENTS

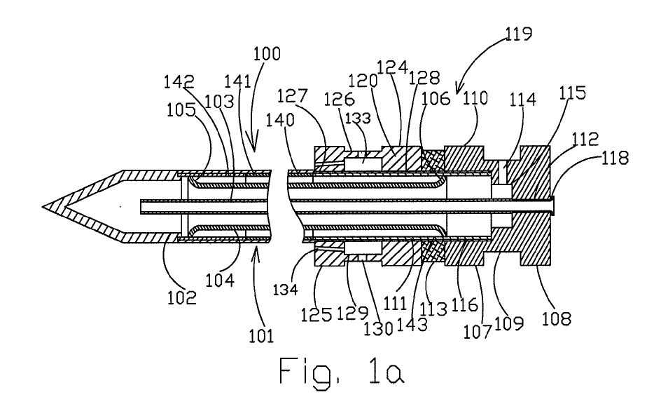

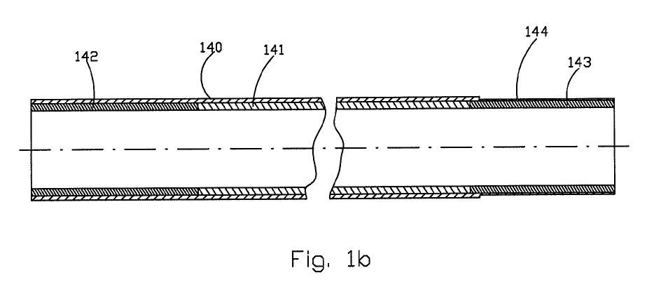

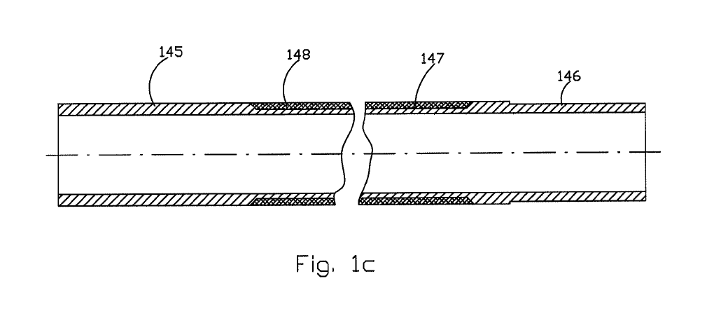

FIG. 1 shows an axial cross-section of a cryosurgical instrument 100 according to the present invention with heating an external shaft by streams of a gaseous medium, which preferably have a high water content (ie are humidified). This embodiment of cryosurgical instrument 100 comprises the elongated external shaft 101, which terminates at its distal edge with cryotip 102.

There is a central feeding pipe 103, which is situated in shaft 103. Preferably, the proximal end of the central feeding pipe 103 protrudes from the proximal end of shaft 101. The proximal sections of the elongated external shaft 101 and the central feeding pipe 103 serve for installation of a male unit 119 for quick coupling. The extreme proximal section of the shaft 101 preferably protrudes at least partially to prevent movement past a certain point with regard to male unit 119.

Thermal insulation of the elongated external shaft 101 is ensured by an intermediate tube 104 with two flanged ends 105 and 106, wherein the outer diameter of the formed flanges 105 and 106 conforms to the internal diameter of the shaft. Friction between the internal surface of the elongated external shaft 101 and flanging 106 ensures stable positioning of the intermediate tube 104 relative to the elongated external shaft 101.

The male unit 119 of the quick coupling, which is installed on the proximal sections of the elongated external shaft 101 and the central feeding pipe 103, preferably comprises a first bushing 107; the outer and internal surfaces of this first bushing 107 are preferably stepped.

The outer surface of the first bushing 107 preferably comprises proximal and distal cylindrical sections 108 and 110, and a middle section 109; the proximal and distal sections 108 and 110 have the same diameter, while the diameter of the middle section 109 is somewhat smaller.

The inner surface of the first bushing 107 is preferably also stepped: it preferably has distal, intermediate and proximal sections 116, 115 and 112 with progressively reduced diameters.

The first bushing 107 is installed on the proximal sections of shaft 101 and the central feeding pipe 103 such that the distal section of the inner surface of the bushing 107 is fitted tightly on the proximal section of the shaft 101, while the proximal inner surface 112 of bushing 107 is fitted slidingly on the proximal section of the central feeding pipe 103. After positioning the first bushing 107 on the proximal section of the elongated external shaft 101, the proximal edge of the central feeding pipe 103 is preferably flanged with application of a deformable o-ring 118, more preferably constructed from a cryogenically stable polymer, for sealing the gap between the proximal sections of the internal surface of the first bushing 107 and the central feeding pipe 103. There is a first channel 114, which communicates between the internal and external spaces of inner intermediate section 115 and outer middle section 109 of the first bushing 107, for receiving the temperature controlling fluid medium, such as a gas for example.

A second bushing 120 is preferably installed on the longitudinally turned section 111 of the elongated external shaft 101 distally to the first bushing 107 and spaced from this first bushing 107 by a thermo-insulating ring 113.

The outer surface of the second bushing 120 preferably comprises proximal and distal cylindrical sections 124 and 125 and a middle section 126; the proximal and distal sections 124 and 125 have the same diameter, while the diameter of the middle section 126 is somewhat smaller.

In a similar manner, the inner surface of the second bushing 120 preferably comprises proximal and distal cylindrical sections 127 and 128 and a middle section 129; the proximal and distal sections 127 and 128 have the same diameter, while diameter of the middle section 129 is somewhat larger.

There are preferably a plurality of nozzles 130 in the distal section 128 of the male unit 133 of a quick coupling; the axes of these nozzles 130 form acute angles with the axis of the external shaft 101. Nozzles 130 preferably receive a humidified warming gas from an external source through channel 114 (not shown), at a sufficiently high temperature so as to protect the tissue contacted by the instrument (not shown) and then emit this gas onto the tissue.

The external shaft 101 is divided into the proximal, middle and distal sections; these sections preferably include a common outer tubular piece 140, an internal middle tubular piece 141 from a material with high specific thermal conductivity (including but not limited to silver, copper, brass, aluminum and the like), an internal proximal tubular piece 143 from a material with relatively low specific thermal conductivity (including but not limited to stainless steel, titanium and the like) and an internal distal tubular piece 142 from a material with relatively low specific thermal conductivity (including but not limited to stainless steel, titanium and the like). The common outer tubular piece 140 is preferably from a metal with good compatibility with human tissue, for example, stainless steel, silver, titanium, gold or any other suitable material. Thus, the low temperature of cryotip 102 is blocked from moving to the portion of the instrument in contact with the tissue, while the warmer temperature of the portion in contact with the tissue cannot heat cryotip 102.

FIG. 1b is an axial cross-section of an external shaft with a middle internal tubular piece from a material with high specific thermal conductivity.

The external shaft 150 comprises the outer tubular piece 140 with a turned down proximal section 144, which serves for installation a male unit of a quick coupling. This outer tubular piece is fabricated preferably from a metal with good biological compatibility which also has low thermal conductivity (stainless steel, titanium).

In addition, there are an internal distal tubular piece 142, an internal middle tubular piece 141 and a proximal tubular piece 143, which are joined by tightly fitting with the outer tubular piece 140.

FIG. 1c is an axial cross-section of an external shaft with a middle outer coating by a diamond film.

It comprises a tubular piece 145 with a middle turned section 147 and a proximal turned section 146. The middle turned section 147 is coated with a diamond film 148 which is optionally and preferably a thick diamond coating, more preferably at least about 100 microns for example. Diamond film 148 provides excellent thermal conductivity, which is higher than that of metal.

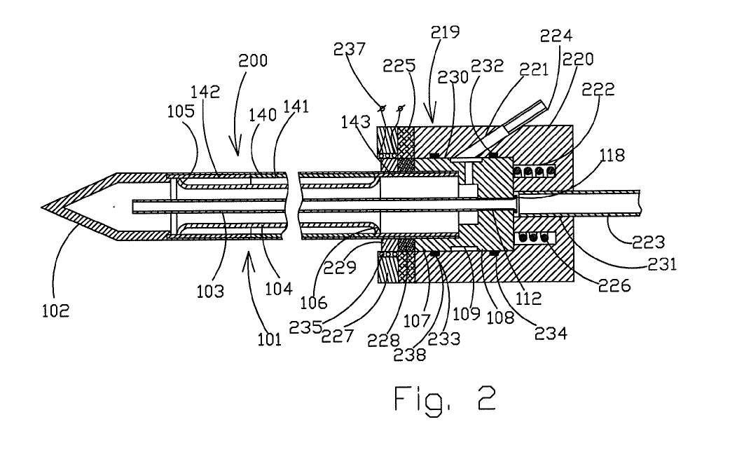

FIG. 2 demonstrates an axial cross-section of the cryosurgical instrument 200 with heating of the external shaft by an outer electrical coil. Elements with the same reference number as for FIG. 1 have the same or at least a similar function.

In addition, the inner surface of the face plane of housing 220 is provided with blind holes 222 and helical springs 226, which are partially situated in these blind holes 222. In such a way, in the process of coupling, the male unit of the coupling pair is spring-actuated by these helical springs 226.

There is a second through channel 221 with an outlet connection 224 installed on the outer end of the second through channel 221, which communicates the annular channel formed between the middle section of bushing 107 and the outside space of housing 220.

In such a way, these annular channels and second through channel 221 serve for exhausting evaporated cryogen from the cryosurgical instrument 200. There is a thermo-insulating ring 228, which is installed on the distal face plane of housing 220, the distal face plane of this thermo-insulating ring 228 serves in turn for installation of ring 227 with an electrical heating spiral 235 on its inner cylindrical surface. Electrical contacts 237 serve for power supply to the electrical heating spiral 235 which therefore heats the external shaft 101.

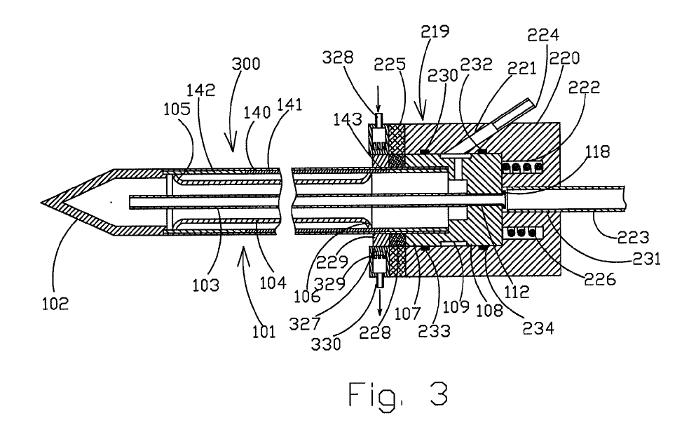

FIG. 3 is an axial cross-section of the cryosurgical instrument 300 with heating the external shaft by a warming fluid medium flowing via an annular chamber. Elements with the same reference number as for FIG. 1 or 2 have the same or at least a similar function.

The distal face plane of thermo-insulating ring 228 serves in this embodiment for installation of an annular heating chamber 327 with an internal radiator 329 on its inner cylindrical surface. The annular heating chamber 327 is provided with an inlet and outlet connections 328 and 330 for delivery and removal of a heating fluid medium into the annular heating chamber 327 which therefore is heating the proximal section of the elongated external shaft 101.

The inner cylindrical surface of the annular heating chamber 327 is provided with fins 329 in order to improve heat transfer between this surface and the heating fluid medium.

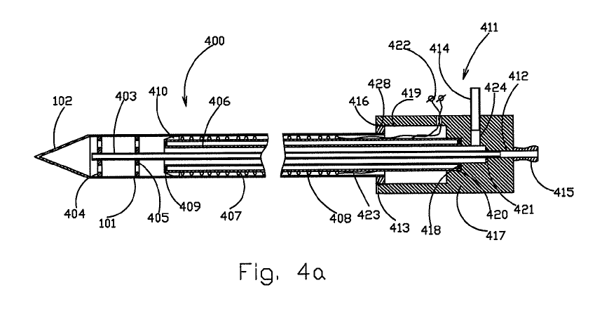

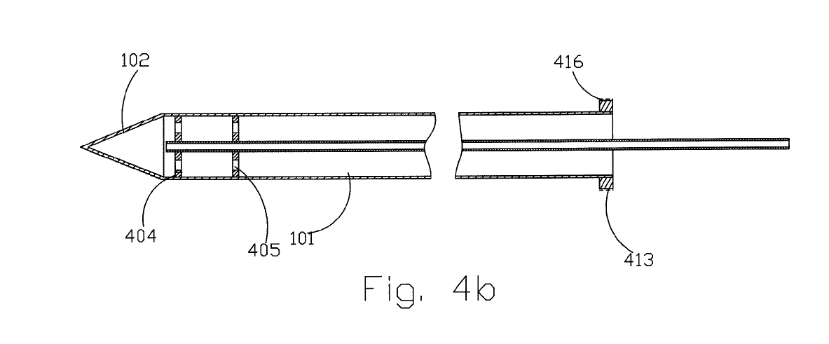

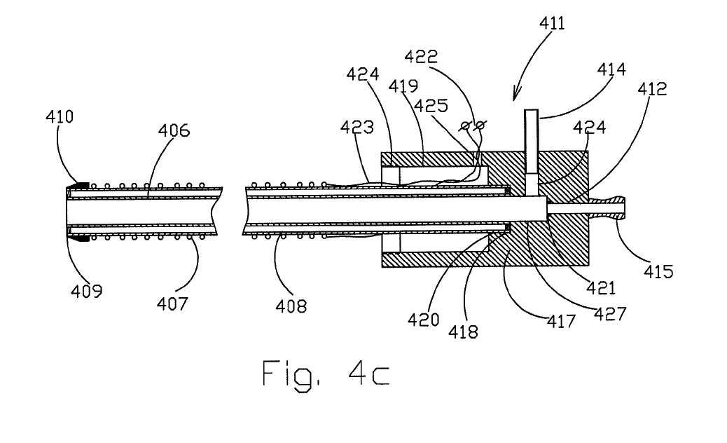

FIG. 4a is an axial cross-section of the cryosurgical instrument 400, which is assembled from a disposable unit comprising an external shaft, a cryotip and a central feeding lumen; and a permanent unit, which comprises a connection element and an elongated electrical heater installed on a cylindrical elongated thermal insulator; FIG. 4b is an axial cross-section of the disposable unit and FIG. 4c is an axial cross-section of the permanent unit of FIG. 4a.

The disposable unit of the cryosurgical instrument comprises: an elongated external shaft 101 with cryotip 102 and a proximal bushing 413, which is installed on the outer side of the elongated external shaft 101 and provided with threading 416. A central feeding lumen 403 is held in the elongated external shaft 101 by two perforated disks 404 and 405.

It should be noted that the central feeding lumen 403 preferably protrudes significantly outside the proximal edge of the elongated external shaft 101.

A permanent unit 411 of the cryosurgical instrument 400 comprises bushing 417 with a distal cylindrical cavity 419 and a distal internal threading 428. A cylindrical surface 420 of a smaller diameter in bushing 417 serves for installation of the proximal section of an outer tubular piece 408 with an elongated electrical heater 407; opening 425 serves for passage of wires 423 of the elongated electrical heater 407 outside bushing 417 with joining these wires 423 with connectors 422. The elongated electrical heater 407 is fabricated from an electrically isolated wire, which is wound on the outer tubular piece 408. In addition, there is a second tubular piece 406, which is joined with the first tubular piece 408 by two sealing rings 409 and 418. The distal end of the outer tubular piece 408 is provided with a stationary bushing 410 in order to protect the elongated electrical heater 407 in the assembling process.

There is a hole 424 in bushing 417 for installation of an outlet connection 414 and a central hole 412, which is in fluid communication with the inlet connection 415. In addition, there is a sealing O-ring 421, which is installed near the edge of the central hole 412. There is a second tubular piece 406, which is joined with the first tubular piece 408 by two sealing rings 409 and 418, the proximal edge of the first tubular piece 408 is installed on the cylindrical surface 420 of bushing 417. The distal end of the first tubular piece 408 is provided with a stationary bushing 410 in order to protect the elongated electrical heater 407 in the assembling process.

A cylindrical surface 427 in the internal cavity of bushing 417, which is of a smaller diameter than the cylindrical surface 420, is provided with hole 424 for installation of an outlet connection 414. There is a central hole 412 in the proximal face plane of bushing 417; this central hole 412 is in fluid communication with the inlet connection 415.

Central feeding lumen 403 receives cryogen which then passes to cryotip 102 for cooling, for example for a cryotherapeutic process, which is preferably a cryosurgical process. To prevent or at least reduce tissue damage, electrical heater 407 heats outer tubular piece 408 to a sufficient extent.

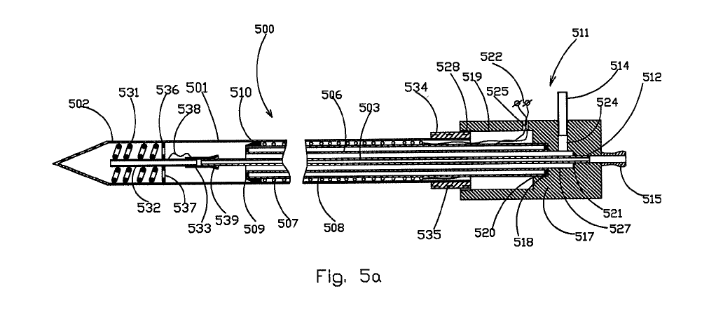

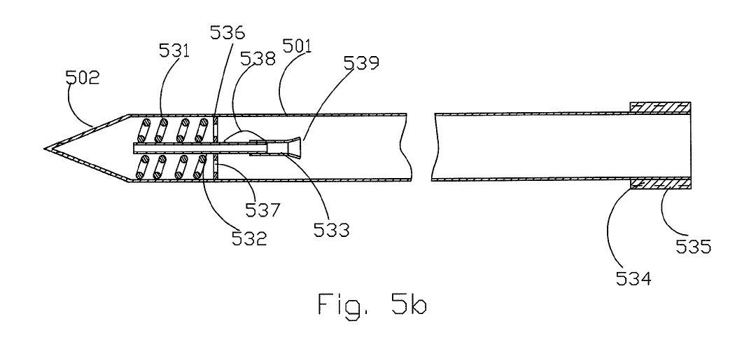

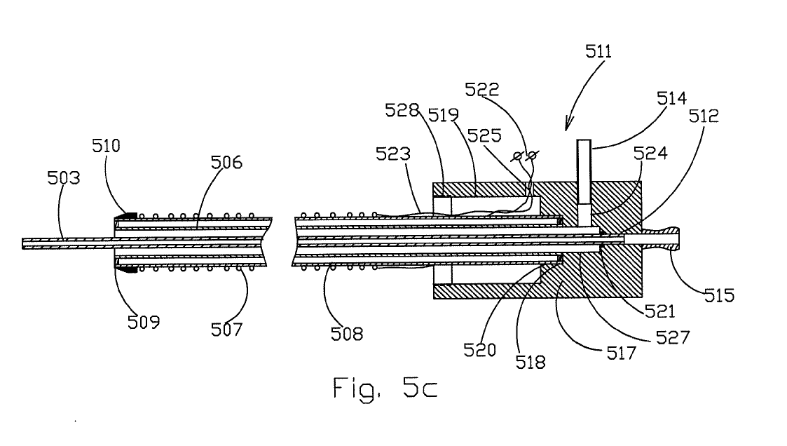

FIG. 5a is an axial cross-section of the cryosurgical instrument 500, which is assembled from a disposable unit comprising an external shaft, a cryotip and a distal member of a central feeding lumen; and a permanent unit, which comprises an inlet and outlet connection elements, an elongated electrical heater installed on a cylindrical elongated thermal insulator and a central feeding lumen; FIG. 5b is an axial cross-section of the disposable unit and FIG. 5c is an axial cross-section of the permanent unit of FIG. 5a.

The disposable unit of the cryosurgical instrument comprises: an elongated external shaft 501 with cryotip 502 and a proximal bushing 528, which is installed on the outer side of the elongated external shaft 501 and provided with threading 534. A distal member of the central feeding lumen is installed in cryotip 502 by disks 536 with holes 537. This distal member comprises a stationary fastened tubular piece 532 with metal spirals 531, which serve for the cryogen droplets' separation. The proximal section of the stationary fastened tubular piece 532 is provided with a displaceable tubular piece 533 with conically flanged edge 539. This displaceable tubular piece 533 is joined with the stationary fastened tubular piece 532 by a wire 538. The displaceable tubular piece 533 with conically flanged edge 539 should be joined in the process of the cryosurgical instrument assembling with the main section 503 of the central feeding lumen, which is installed in the permanent unit 511.

It should be noted that the central feeding lumen 503 preferably protrudes significantly outside the proximal edge of the elongated external shaft 501.

A permanent unit 511 of the cryosurgical instrument 500 comprises bushing 517 with a distal cylindrical cavity 519 and a distal internal threading 528; a cylindrical surface 520 of a smaller diameter serves for installation of the proximal section of a first tubular piece 508 with an binary wire spiral 507, which serves as an electrical heater; opening 525 serves for passage of wires 523 of the binary wire spiral 507 outside bushing 517 with joining these wires 523 with connectors 522. The binary wire spiral 507 is provided with a layer of electrical isolation.

There is a second tubular piece 506, which is joined with the first tubular piece 508 by two sealing rings 509 and 518. The distal end of the first tubular piece 508 is provided with a stationary bushing 510 in order to protect the binary wire spiral 507 in the assembling process.

In addition, the face plane of bushing 517 is provided with a through opening 524 for installation of an outlet connection 514, which serves for removal of exhausted gases, and a central hole 512, which serves for installation of the proximal section of the central feeding lumen 503 and is in fluid communication with the inlet connection 515.

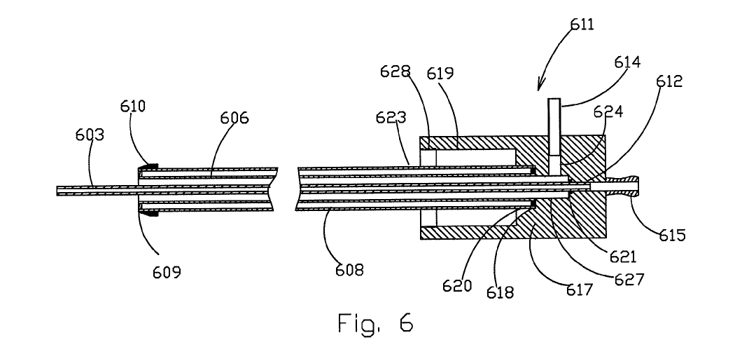

FIG. 6 is an axial cross-section of a permanent unit of a cryosurgical instrument with an elongated vacuum insulated chamber applied instead of the electrical heater. A disposable unit of this cryosurgical instrument has the same design as in FIG. 5b. A permanent unit 611 of the cryosurgical instrument comprises bushing 617 with a distal cylindrical cavity 619 and a distal internal threading 628; a cylindrical surface 620 of a smaller diameter serves for installation of the proximal section of a tubular piece 608.

There is a second thermo-insulating tubular piece 606, which is joined with the first thermo-insulating tubular piece 608 by two sealing rings 609 and 618, and air in the interior between these first and second tubular pieces 608 and 606 is evacuated to form a vacuum.

In addition, the face plane of bushing 617 is provided with a through opening 624 for installation of an outlet connection 614, which serves for removal of exhausted gases, and a central hole 612, which serves for installation of the proximal section of the central feeding lumen 603 and is in fluid communication with the inlet connection 615.

While the invention has been described with respect to a limited number of embodiments, it will be appreciated that many variations, modifications and other applications of the invention may be made.

1. A cryosurgical instrument, comprising:
an inner lumen for receiving a cryogen, said inner lumen terminating at or before a cryotip;
an external shaft for containing said inner lumen and terminating at said cryotip; wherein said external shaft is made from a metal or from another material with sufficiently high specific thermal conductivity; and
a heating means, which is installed on another proximal section of said external shaft for ensuring positive temperatures of the external surface of said external shaft in the process of its operation at the outer section of said shaft and at a certain deepness from the place of penetration of said cryosurgical instrument into a tissue of a body.

2. The instrument of claim 1, further comprising an intermediate lumen between said inner lumen and said external shaft, said intermediate lumen terminating before said cryotip; wherein a gas-filled gap between said intermediate lumen and said external shaft comprises a thermo-insulating layer.

3. The instrument of claim 2, further comprising a coupling means, installed on the outer proximal section of said external shaft and including at least an inlet connection for supply of said cryogen into said inner lumen and an outlet connection for removal the exhausted liquid-gaseous cryogen from the inner space of said cryosurgical instrument.

4. The instrument of claim 3, wherein the cryosurgical instrument operates on the basis of liquid or liquid-gaseous cryogen, which is supplied into the proximal end of the inner lumen and is boiling on the inner surface of the cryotip.

5. The instrument of claim 3, wherein the cryosurgical instrument operates on the basis of adiabatic expansion of highly pressurized gas (Joule-Thomson principle); wherein the inner lumen is designed as a counter-flow heat exchanger and the distal end of said inner lumen is provided with an orifice.

6. The instrument of claim 3, wherein the heating means comprises an annular chamber with an inlet connection, said annular chamber being installed distally to the coupling means and in immediate vicinity to said coupling means; wherein the distal face plane of said annular chamber is provided with a set of nozzles directed at a certain angle to the axis of the external shaft; wherein said inlet connection supplies a pressurized heating gaseous medium into said annular chamber, and wherein said nozzles force said pressurized heating gaseous medium into said external shaft.

7. The instrument of claim 3, wherein the heating means comprises an annular chamber with an inlet connection, said annular chamber being installed distally to the coupling means and in immediate vicinity to said coupling means; said annular chamber comprising an outlet connection; wherein a heating medium is supplied into and out of said annular chamber and immediate thermal contact of the inner cylindrical surface of said annular chamber with the proximal section of said external shaft induces heat transfer.

8. The instrument of claim 3, wherein the heating means is designed as a metal ring placed distally to the coupling means and in immediate vicinity to said coupling means; said metal ring is heated by an electrical heater installed in a female unit of said coupling means.

9. The instrument of claim 8, wherein the electrical heater is an electrical resistor.

10. The instrument of claim 8, wherein the electrical heater is an electrical inductor.

11. The instrument of claim 8, wherein said cryosurgical instrument comprises two units to be assembled before its usage: a disposable unit, which consists of : an elongated external shaft with cryotip and a proximal bushing, which installed on the outer side of said elongated external shaft and provided with threading; a central feeding lumen is installed in said elongated external shaft by two perforated disks;
and a permanent unit of said cryosurgical instrument comprises: a bushing with a distal cylindrical step-wise cavity and a distal internal threading; there are two coaxial tubular pieces, the proximal section of the outer tubular piece is installed in a cylindrical inner surface of said bushing and the edge of said coaxial tubular pieces are mutually sealed; an elongated electrical heater with electrical isolation is installed on the surface of said outer tubular; an opening in said bushing serves for passage of wires of said elongated electrical heater outside said bushing with joining said wires with electrical connectors; and
a proximal cylindrical surface of said internal cavity of said bushing is provided with a hole for installation of an outlet connection, and a central hole, which is in fluid communication with an inlet connection, serves for installation of an inlet connection.

12. The instrument of claim 11, wherein the disposable unit of said cryosurgical instrument comprises a distal section of the central feeding lumen and the permanent unit of said cryosurgical instrument comprises the main section of said central feeding lumen.

13. The instrument of claim 11, wherein the interior between the coaxial tubular pieces features a vacuum.

14-18. (canceled)
